(19) United States
(12) Patent Application Publication
(10) Pub. No.: US 2025/0312018 A1
(43) Pub. Date: Oct. 9, 2025

(54) BIOLOGICAL SAMPLE COLLECTION SYSTEM WITH PUSH-ACTIVATED VALVE FOR SAMPLE RELEASE

(71) Applicant: DNA Genotek Inc., Kanata, Ontario (CA)

(72) Inventors: Attila Csaba NEMETH, White Lake (CA); Adele JACKSON, Stittsville (CA); Karina Gail CASTILLA, Carleton Place (CA)

(73) Assignee: DNA Genotek Inc., Kanata, Ontario (CA)

(21) Appl. No.: 18/865,083

(22) PCT Filed: May 10, 2023
(86) PCT No.: PCT/CA2023/050641

§ 371 Date: Nov. 12, 2024

Related U.S. Application Data
(60) Provisional application No. 63/340,396, filed on May 10, 2022.

(51) Int. Cl. A61B 10/00; B01L 3/00

(52) U.S. Cl. CPC A61B 10/0051; B01L 3/502; B01L 2300/042; B01L 2300/0672; B01L 2400/0655; B01L 2400/0683

(57) ABSTRACT

A biological sample collection system including a containment vessel; a sample collection reservoir disposed toward a first end of the containment vessel and a valve disposed in a sample receiving area. The valve includes a closed configuration, such that when the valve is in the closed configuration the biological sample is retained in the sample collection reservoir, and an open configuration, such that when the valve is in the open configuration the biological sample is released from the sample collection reservoir into a sample storage chamber of the containment vessel. A cap includes a pusher. When the biological sample collection system is closed by engagement of the containment vessel with the cap, the pusher engages with the valve thereby moving the valve from the closed configuration to the open configuration to permit the biological sample to be released from the sample collection reservoir into the containment vessel.

### FIELD

The present invention pertains to a biological sample collection system. More particularly, the present invention pertains to a biological sample collection system with a push-activated valve for sample release.

### BACKGROUND

Field collections of biological samples, such as saliva or sputum, can provide invaluable information about the patient or donor. It can be inconvenient to require patients/donors to travel to a biological sample collection site or laboratory. Similarly, it may be difficult and costly for laboratory personnel to directly access the patient/donor for sample collection, particularly if the sample size is large and/or geographically diverse. In addition, patients/donors are often inexperienced, first-time donors, so it is critical that the biological sample collection system be easy to operate or manipulate by novice users. A desirable collection system removes the need for specialized personnel/clinicians, facilities and equipment when collecting, transporting and storing biological samples.

At ambient temperature, nucleic acids and other biomolecules (e.g. metabolites) in biological samples quickly degrade and must generally be stored under freezing temperatures to remain stable. This problem is amplified when a biological sample is collected at a remote field site, or a significant distance from the laboratory or doctor's office, and especially where power and freezers are non-existent or not constant. In addition, shipping biological samples on dry ice or ice packs is costly, complicated, and impractical and could lead to the exposure of workers to infectious agents or pathogens. Hence, there is a need for a safe collection, transport and storage system that maintains the integrity of the collected biomolecules in biological samples under ambient conditions for further molecular analysis or diagnostic testing.

There is a need for an improved container system for releasably and reliably storing a substance. It is often desirable to store a substance, such as a liquid, solid, slurry, gas, mixtures thereof, or the like, in a container or compartment prior to mixing the contents of the container with another material. For example, it may be desirable to package and store a compound, or compounds, in a container for shipping and/or safe storage and handling, prior to combining the compound(s) with another material.

It may be desirable to package and store a toxic compound in a container, prior to combining such a toxic compound with a detoxifying material or reagent. As well, it is often desirable to keep a concentrated active ingredient separate from a diluent until immediately prior to use. Container systems having a separate, sealed compartment/chamber for the sample preservative protect the patient/specimen donor from contacting, ingesting and/or spilling the preservative. Safely confining the toxic compound or preservative within a separate compartment of the sample collection device or system allows the use of more potent chemicals or preservatives (e.g. denaturants) to stabilize biomarkers/biomolecules, e.g. nucleic acids and metabolites, contained in the collected specimen. Only after the device or system containing the biological specimen is closed with a lid or cap does the compartment for the preservative open and allow mixing of the biological specimen with the preservative.

There are a variety of containers/systems for holding substances separately in such a manner that a user may seal a closure to combine the substances. Typically, these containers are double compartment systems in which substances are stored separately and substances are combined by application of the container/system closure by a user. There are numerous ways to design said two compartment/chamber systems. In particular, there is a need for a patient-friendly sample collection device or system that allows for safe at-home collection, followed by leak-proof transportation and storage under or ambient room temperature conditions. Two compartment/chamber systems are ideal for protecting the donor/patient from the preservative needed to preserve the sample or analyte of interest until analysis. Ideally, the method or instructions involve a minimal number of steps for use.

There is a need for biological sample collection systems to facilitate sample collection.

This background information is provided for the purpose of making known information believed by the applicant to be of possible relevance to the present invention. No admission is necessarily intended, nor should be construed, that any of the preceding information constitutes prior art against the present invention.

### SUMMARY

In one aspect, there is provided a biological sample collection system, the system comprising: (a) a containment vessel comprising: a first end for receiving a biological sample, a second end comprising a sample storage chamber, and a connection member disposed at the first end; (b) a sample collection reservoir disposed toward the first end of the containment vessel, wherein the sample collection reservoir comprises: an opening for receiving the biological sample, one or more walls defining a sample receiving area, and a valve disposed in the sample receiving area, the valve comprising: a closed configuration, wherein when the valve is in the closed configuration the biological sample is retained in the sample collection reservoir, and an open configuration, wherein when the valve is in the open configuration the biological sample is released from the sample collection reservoir into the sample storage chamber of the containment vessel; and (c) a cap comprising: a top end, an open end, a pusher comprising a first end extending from an inner portion of the cap and an opposing second end, the second end of the pusher extending a distance from the inner portion of the cap towards the open end of the cap and being configured to engage with the valve of the sample collection reservoir, and a connection member complementary to the connection member of the containment vessel and configured to engage the open end of the cap with the first end of the containment vessel; wherein, when the biological sample collection system is closed by engagement of the connection member of the containment vessel with the connection member of the cap, the second end of the pusher engages with the valve of the sample collection reservoir, thereby moving the valve from the closed configuration to the open configuration to permit the biological sample to be released from the sample collection reservoir into the sample storage chamber of the containment vessel.

In another aspect, there is provided a method of preserving a biomolecule in a biological sample, the method comprising: a) obtaining a biological sample; b) obtaining the biological sample collection system as described herein; c) placing the biological sample in the sample collection reservoir; d) placing the cap over the first end of the containment vessel; e) engaging the connection member of the containment vessel with the connection member of the cap, which engages the second end of the pusher with the valve of the sample collection reservoir, thereby moving the valve from the closed configuration to the open configuration to permit the biological sample to be released from the sample collection reservoir into the sample storage chamber of the containment vessel; and f) mixing the released biological sample with a stabilizing composition in the containment vessel for preserving the biomolecule within the biological sample.

### BRIEF DESCRIPTION OF THE FIGURES

For a better understanding of the present invention including the progression of development to get to the end product, reference is made to the following description which is to be used in conjunction with the accompanying drawings, where:

FIG. 1 illustrates a perspective, exploded view of a biological sample collection system 100 in accordance with one embodiment of the present application.

FIG. 2a illustrates a side, exploded view of the biological sample collection system 100 of FIG. 1.

FIG. 2b is a simplified cross-sectional view taken along line B-B of FIG. 2a.

FIG. 2c illustrates a perspective view of the sample collection reservoir 112 inserted into the tube 102 of the biological sample collection system 100 of FIG. 1.

FIG. 3a illustrates a side view of the assembled biological sample collection system 100 of FIG. 1.

FIG. 3b shows a simplified cross-sectional view taken along line D-D of FIG. 3a.

FIG. 3c is an enlarged view of area F as shown in FIG. 3b.

FIG. 4a illustrates a perspective view of the cap 126 of the biological sample collection system 100 of FIG. 1.

FIG. 4b illustrates a bottom view of cap 126 of the biological sample collection system 100 of FIG. 1.

FIG. 5a illustrates a perspective view of the sample collection reservoir 112 of the biological sample collection system 100 of FIG. 1;

FIG. 5b illustrates a top view;

FIG. 5c illustrates a bottom view;

FIG. 5d illustrates a side view; and

FIG. 5e is a cross-sectional view of the sample collection reservoir 112 taken along line G-G of FIG. 5d.

FIG. 5f illustrates a perspective view of an alternate embodiment of the sample collection reservoir 112 of the biological sample collection system 100 of FIG. 1, wherein the sample collection reservoir comprises markings which correspond to a fluid volume in the sample receiving area.

FIGS. 6a-c illustrate a simplified cross-sectional view of an alternate biological sample collection system 100′. FIG. 6a illustrates a simplified cross-sectional view of the alternate biological sample collection system 100′ with the cap 126′ separated from the tube 102′, and the sample collection reservoir 112′ disposed toward the first end 104′ of the tube 102′ with the valve 120′ in the closed configuration.

FIG. 6b illustrates a simplified cross-sectional view of the biological sample collection system 100′ wherein the biological sample collection system 100′ is closed and the valve 120′ is in the open configuration.

FIG. 6c is an enlarged view of the ring/snap feature that secures the sample collection reservoir 112′ to the tube 102′.

FIGS. 7a-c illustrate a simplified cross-sectional view of an alternate biological sample collection system 100″. FIG. 7a illustrates a simplified cross-sectional view of the alternate biological sample collection system 100″ with the cap 126″ separated from the tube 102″, and the sample collection reservoir 112″ disposed toward the first end 104″ of the tube 102″ with the valve 120″ in the closed configuration.

FIG. 7b illustrates a simplified cross-sectional view of the biological sample collection system 100″ wherein the biological sample collection system 100″ is closed and the valve 120″ is in the open configuration.

FIG. 7c is an enlarged view of the lip/shoulder engagement that secures the sample collection reservoir 112″ to the tube 102″.

FIG. 8a illustrates a side, exploded view of an alternate biological sample collection system 100a.

FIG. 8b shows a simplified cross-sectional view taken along line D-D of FIG. 8a.

FIG. 9a illustrates a side, exploded view of the biological sample collection system 100a of FIG. 8a, showing the cap 126a and showing the sample collection reservoir 112a inserted into the tube 102a.

FIG. 9b shows a simplified cross-sectional view taken along line A-A of FIG. 9a.

FIG. 10a illustrates a side view of the assembled biological sample collection system 100a of FIG. 8a, in an initial shipping configuration with the cap 126a partly engaged with the tube 102a.

FIG. 10b shows a simplified cross-sectional view taken along line B-B of FIG. 10a.

FIG. 11a illustrates a side view of the assembled biological sample collection system 100a of FIG. 8a, in a closed configuration following sample collection with the cap 126a fully engaged with the tube 102a.

FIG. 11b shows a simplified cross-sectional view taken along line C-C of FIG. 11a.

FIG. 12a illustrates a perspective view of the cap 126a of the biological sample collection system 100a of FIG. 8a;

FIG. 12b shows a side view;

FIG. 12c shows a top view; and FIG. 12d shows a bottom view.

FIG. 13a illustrates a perspective view of the sample collection reservoir 112a of the biological sample collection system 100a of FIG. 8a;

FIG. 13b shows a side view;

FIG. 13c shows a cross-sectional view taken along line E-E of FIG. 13b;

FIG. 13d shows an enlarged view of area F as shown in FIG. 13c;

FIG. 13e shows a top view; and

FIG. 13f shows a bottom view of the sample collection reservoir 112a.

FIG. 14 illustrates a side, exploded view of an alternate biological sample collection system 100b.

FIG. 15a illustrates a side, exploded view of the biological sample collection system 100b of FIG. 14, showing the cap 126b and showing the sample collection reservoir 112b inserted into the tube 102b.

FIG. 15b shows a simplified cross-sectional view taken along line B-B of FIG. 15a.

FIG. 16a illustrates a side view of the assembled biological sample collection system 100b of FIG. 14, in an initial shipping configuration with the cap 126b partly engaged with the tube 102b.

FIG. 16b shows a simplified cross-sectional view taken along line C-C of FIG. 16a.

FIG. 16c is an expanded view of a portion of FIG. 16b illustrating the interaction between the pusher 132b of the cap 126b and the valve 120b when the cap 126b is partly engaged with the tube 102b.

FIG. 17a illustrates a side view of the assembled biological sample collection system 100b of FIG. 14, in a closed configuration following sample collection with the cap 126b fully engaged with the tube 102b.

FIG. 17b shows a simplified cross-sectional view taken along line D-D of FIG. 17a.

FIG. 17c is an expanded view of a portion of FIG. 17b illustrating the interaction between the pusher 132b of the cap 126b and the valve 120b when the cap 126b is fully engaged with the tube 102b.

FIG. 18a illustrates a side view of the cap 126b of the biological sample collection system 100b of FIG. 14;

FIG. 18b shows a simplified cross-sectional view taken along line T-T of FIG. 18a;

FIG. 18c shows a top view; and

FIG. 18d shows a bottom view.

FIG. 19a illustrates a perspective view of the sample collection reservoir 112b of the biological sample collection system 100b of FIG. 14;

FIG. 19b shows a side view;

FIG. 19c shows a cross-sectional view taken along line R-R of FIG. 19b; and

FIG. 19d shows a top view.

### DETAILED DESCRIPTION OF THE INVENTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) ingredient(s) and/or elements(s) as appropriate.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

The term “bodily fluid” as used herein will be understood to mean a naturally occurring fluid from a human or an animal, and includes, but is not limited to urine, saliva, sputum, serum, plasma, blood, pharyngeal, nasal/nasal pharyngeal and sinus secretions, mucous, gastric juices, pancreatic juices, bone marrow aspirates, cerebral spinal fluid, feces, semen, products of lactation or menstruation, cervical secretions, vaginal fluid, tears, or lymph. In one embodiment, the bodily fluid is selected from sputum or saliva.

The term “ambient temperature” as used herein refers to a range of temperatures that could be encountered by the biological sample collection system as described herein, which can contain a mixture of a bodily fluid (e.g. saliva sample) and a stabilizing composition, from the point of collection, during transport (which can involve relatively extreme temperatures, albeit usually for shorter periods of time (e.g. <5 days)), as well as during prolonged storage prior to analysis. In one embodiment, the ambient temperature is ranging from about −20° C. to about 50° C. In another embodiment, the ambient temperature is room temperature (RT) and ranges from about 15° C. to about 25° C.

As noted above, two compartment/chamber systems are ideal for protecting the donor/patient from the preservative needed to preserve the sample or analyte of interest until analysis. One mechanism to separate two compartments within a collection system is the utilization of a valve. The sealing mechanism of a sample collection device may comprise a one-way valve or check valve which allows fluid (liquid or gas) to flow through it in only one direction. There are various types of check valves used in a wide variety of applications. An example of a common one-way valve is a duckbill valve. These valves are often manufactured from rubber or synthetic elastomer and have two or more flaps. The application of pressure causes the flattened duckbill flaps to open to permit the pressurized fluid (or gas) to pass. When pressure is removed, the duckbill valve returns to its flattened shape, preventing backflow. Other examples of one-way valves includes ball check valves, diaphragm check valves, a swing or tilting check valve, a flapper or clapper valve, a lift-check valve, an in-line check valve and a flow check valve.

As outlined in further detail below, the system as described herein is a two compartment/chamber system that makes use of a valve to separate a sample collection reservoir from a sample storage chamber, wherein the valve is moved to an open position by mechanical coupling of the valve with a cap of the system following collection of a biological sample.

In one embodiment, there is provided a biological sample collection system, the system comprising: (a) a containment vessel comprising: a first end for receiving a biological sample, a second end comprising a sample storage chamber, and a connection member disposed at the first end; (b) a sample collection reservoir disposed toward the first end of the containment vessel, wherein the sample collection reservoir comprises: an opening for receiving the biological sample, one or more walls defining a sample receiving area, and a valve disposed in the sample receiving area, the valve comprising: a closed configuration, wherein when the valve is in the closed configuration the biological sample is retained in the sample collection reservoir, and an open configuration, wherein when the valve is in the open configuration the biological sample is released from the sample collection reservoir into the sample storage chamber of the containment vessel; and (c) a cap comprising: a top end, an open end, a pusher comprising a first end extending from an inner portion of the cap and an opposing second end, the second end of the pusher extending a distance from the inner portion of the cap towards the open end of the cap and being configured to engage with the valve of the sample collection reservoir, and a connection member complementary to the connection member of the containment vessel and configured to engage the open end of the cap with the first end of the containment vessel; wherein, when the biological sample collection system is closed by engagement of the connection member of the containment vessel with the connection member of the cap, the second end of the pusher engages with the valve of the sample collection reservoir, thereby moving the valve from the closed configuration to the open configuration to permit the biological sample to be released from the sample collection reservoir into the sample storage chamber of the containment vessel.

In another embodiment, the connection members comprise a threaded connection.

In yet another embodiment, engagement of the connection member of the containment vessel with the connection member of the cap creates a fluid-tight seal.

In still yet another embodiment, the sample collection reservoir comprises at least one marking on said at least one or more walls which corresponds to a fluid volume in the sample receiving area.

In another embodiment, the sample collection reservoir is funnel-shaped.

In another embodiment, the valve is a duck-bill valve, and when the biological sample collection system is closed by engagement of the connection member of the containment vessel with the connection member of the cap, the second end of the pusher engages with and extends through the duck-bill valve to move the valve from the closed configuration to the open configuration.

In yet another embodiment, the valve is formed from a flexible material or a rigid plastic having at least one pre-scored cut, wherein the at least one pre-scored cut does not fully penetrate through the flexible material or rigid plastic, and when the biological sample collection system is closed by engagement of the connection member of the containment vessel with the connection member of the cap, the second end of the pusher engages with the at least one pre-scored cut to penetrate and extend through the flexible material or rigid plastic to move the valve from the closed configuration to the open configuration.

In another embodiment, the pusher comprises a groove in an outer surface thereof, wherein the groove extends along at least a portion of the second end of the pusher along a longitudinal axis of the pusher. In another embodiment, the groove extends from the first end of the pusher to the second end of the pusher.

In yet another embodiment, the valve has a valve seat and a valve face, wherein the valve face is movable with respect to the valve seat between the closed configuration, in which the valve seat sealingly engages the valve face, and the open configuration, in which the valve face is spaced from the valve seat to allow fluid flow between the valve face and the valve seat, and when the biological sample collection system is closed by engagement of the connection member of the containment vessel with the connection member of the cap, the second end of the pusher engages with the valve to move the valve face with respect to the valve seat from the closed configuration to the open configuration.

In still yet another embodiment, the sample collection reservoir is connected toward the first end of the containment vessel via a press-fit connection between a lip extending from the one or more walls of the sample collection reservoir and a complementary groove disposed at the first end of the containment vessel.

In another embodiment, the sample collection reservoir is connected toward the first end of the containment vessel via a snap-fit connection between a lip extending from the one or more walls of the sample collection reservoir into a complementary groove disposed on an inner surface of the containment vessel.

In yet another embodiment, the sample collection reservoir is connected toward the first end of the containment vessel via a press-fit connection between a lip extending from the one or more walls of the sample collection reservoir and a shoulder on an inner surface of the containment vessel.

In another embodiment, the sample storage chamber comprises a stabilization composition.

In another embodiment, a method of preserving a biomolecule in a biological sample is provided, the method comprising: a) obtaining a biological sample; b) obtaining the biological sample collection system as described herein; c) placing the biological sample in the sample collection reservoir; d) placing the cap over the first end of the containment vessel; e) engaging the connection member of the containment vessel with the connection member of the cap, which engages the second end of the pusher with the valve of the sample collection reservoir, thereby moving the valve from the closed configuration to the open configuration to permit the biological sample to be released from the sample collection reservoir into the sample storage chamber of the containment vessel; and f) mixing the released biological sample with a stabilizing composition in the containment vessel for preserving the biomolecule within the biological sample.

In another embodiment of the above-described sample collection system, the top end of the cap comprises a second open end (double-ended cap), the second open end being separated from the open end of the cap by the inner portion of the cap, the inner portion of the cap comprising an inner cap wall separating the second open end of the cap from the open end of the cap, the first end of the pusher extending from the inner cap wall, wherein the second open end of the cap comprises a second connection member complementary to the connection member of the containment vessel.

In another embodiment, a method of preserving a biomolecule in a biological sample is provided, the method comprising: a) obtaining a biological sample; b) obtaining the biological sample collection system as described above wherein the top end of the cap comprises a second open end (double-ended cap), wherein the second open end of the cap is connected to the containment vessel via engagement of the second connection member of the cap with the connection member of the containment vessel; c) disengaging the second connection member of the cap from the connection member of the containment vessel, and removing the cap from the containment vessel; d) placing the biological sample in the sample collection reservoir; e) placing the open end of the cap over the first end of the containment vessel, such that the cap is inverted relative to placement of the cap in step (b); f) engaging the connection member of the containment vessel with the connection member of the cap, which engages the second end of the pusher with the valve of the sample collection reservoir, thereby moving the valve from the closed configuration to the open configuration to permit the biological sample to be released from the sample collection reservoir into the sample storage chamber of the containment vessel; and g) mixing the released biological sample with a stabilizing composition in the containment vessel for preserving the biomolecule within the biological sample.

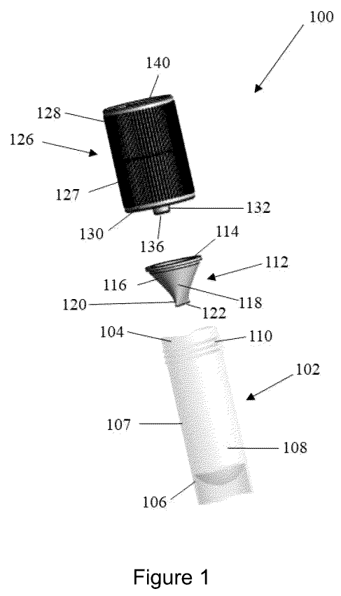

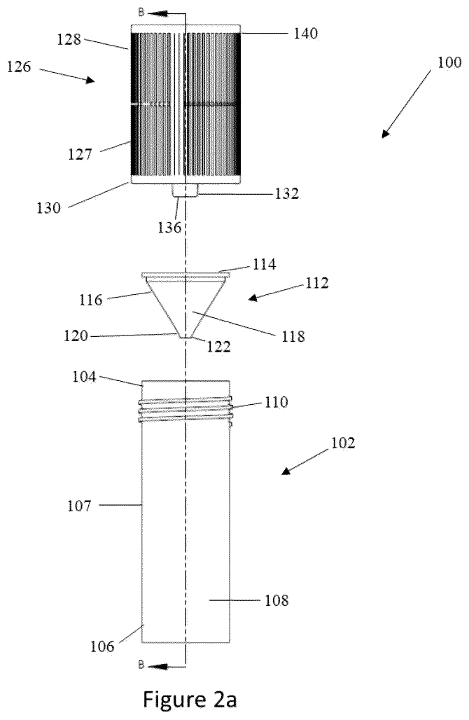

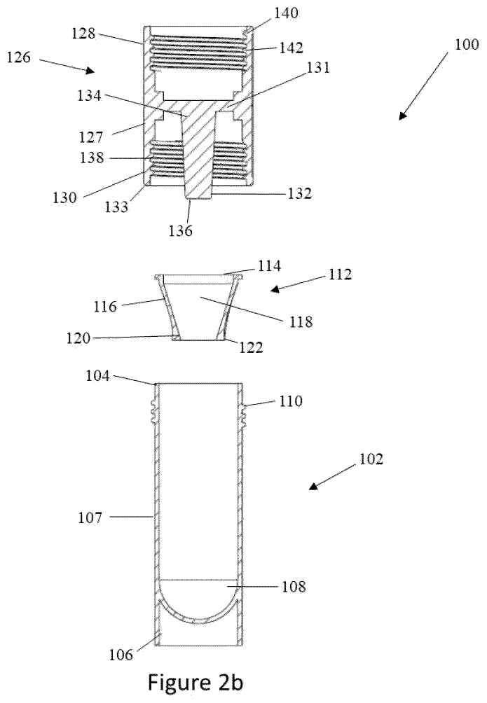

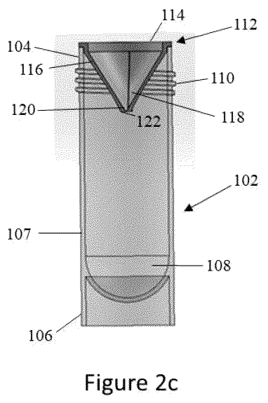

FIGS. 1, 2a-c, 3a-c, 4a-b and 5a-f illustrate a biological sample collection system 100 and components thereof in accordance with one embodiment of the present application. The system 100 includes a containment vessel shown as tube 102, which in the embodiment shown is generally cylindrical in shape. Any suitable containment vessel can be used, such as common sample collection tubes known in the art, or other vials/tubes/containers/vessels having the attributes described herein. The tube 102 comprises a first end 104 for receiving a biological sample, such as a bodily fluid (e.g. sputum or saliva), a second end 106 comprising a sample storage chamber 108 defined by a wall 107. As outlined in further detail below, the sample storage chamber 108 of the tube 102 can contain a stabilizing composition for stabilizing, preserving, and/or facilitating the recovery of biomolecules from the biological sample, which is present in the sample storage chamber 108 prior to sample collection. The tube 102 further comprises a connection member 110 disposed at the first end 104, which in the illustrated embodiment is external helical threads on an outer surface of wall 107 of the tube 102.

The tube 102 can be of any desired width, length or thickness as needed, and is made of an inert, durable material, such as polyethylene, polypropylene, polystyrene or related plastic. The tube 102 is ideally self-standing. The sample storage chamber 108 is suitable for holding a substance such as a liquid, solid, semi-solid, slurry, suspension, powder, colloid, gel, gas, mixtures thereof or the like. The sample storage chamber 108 should have a sufficient void volume to hold the biological sample, plus any desired composition for mixing with the sample.

The wall 107 of the tube 102 should ideally be transparent or translucent to permit viewing of the sample once collected. The wall 107 can be free of any indicia or other markings, and should be suitable to be comfortably handled by the user. However, it may be adorned, if desired, and/or have a grip or a raised texture on an external surface thereof to facilitate handling, or with graduated markings to indicate volume.

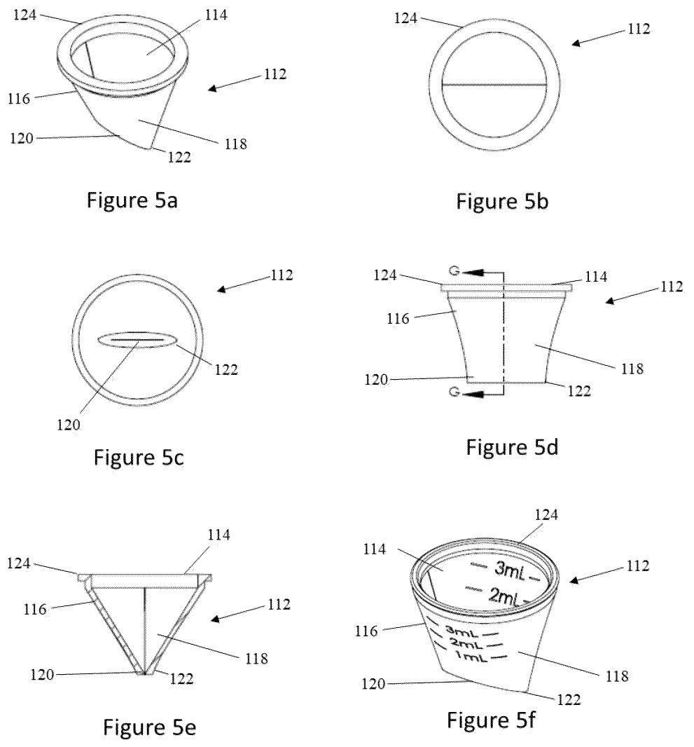

The system 100 further includes a sample collection reservoir 112 that is configured to be disposed toward the first end 104 of the tube 102. FIG. 5a illustrates a perspective view of the sample collection reservoir 112. Top and bottom views of the sample collection reservoir 112 are shown in FIGS. 5b and 5c, respectively. FIG. 5d illustrates a side view of the sample collection reservoir 112, and FIG. 5e is a cross-sectional view of the sample collection reservoir 112 taken along line G-G of FIG. 5d. FIG. 5f illustrates a perspective view of an alternate embodiment of the sample collection reservoir 112 of the biological sample collection system 100 of FIG. 1, wherein the sample collection reservoir comprises markings on the walls 116 which correspond to a fluid volume in the sample receiving area.

The sample collection reservoir 112 has an opening 114 for receiving the biological sample, one or more walls 116 defining the opening 114 and a sample receiving area 118, and a valve 120 disposed in the sample receiving area. In the embodiment shown in FIGS. 1, 2a, 2b, 3b, and 5a-f, the valve 120 is disposed at an end 122 of the sample collection reservoir 112 that is opposite from the opening 114 for receiving the biological sample. The valve 120 as shown in FIGS. 1, 2a, 2b, 3b, and 5a-f is a duck-bill valve, and can be formed from rubber, synthetic elastomer, or another suitable resilient and/or flexible material. In one embodiment, the sample collection reservoir 112 is funnel-shaped, which facilitates collection of the biological sample through the opening 114 and flow of the biological sample to the opposite end 112 of the sample collection reservoir 112, where the valve 120 is located.

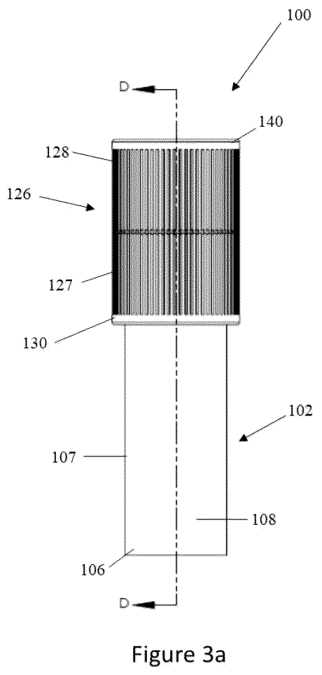

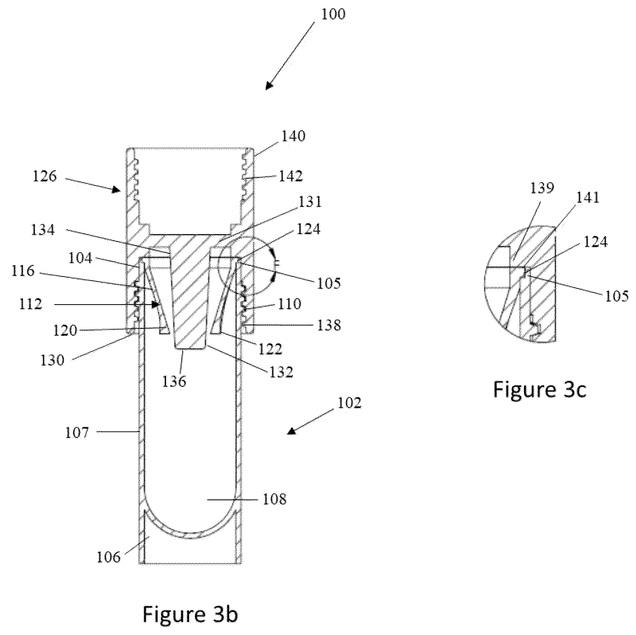

As can best be seen in FIGS. 2c, 3b and 3c, the sample collection reservoir 112 is disposed at the first end 104 of the tube 102. The sample collection reservoir 112 is not intended to be removed from the tube 102 by the user collecting the biological sample, and thus the sample collection reservoir 112 is fitted closely to the tube 102, such as via a snap-fit or press-fit arrangement (e.g. lip and groove). As shown in FIG. 3c, a lip 124 extending from the one or more walls 116 of the sample collection reservoir 112 (proximal to the opening 114) extends into a complementary groove 105 disposed at the first end 104 of the tube 102 to hold the sample collection reservoir 112 in place at the first end 104 of the tube 102. Other fastening arrangements known to the skilled worker, such as gluing, welding, or a threaded engagement, are also contemplated. The sample collection reservoir 112 can also be positioned lower in the tube 102, as described further below with respect to embodiments 100′ and 100″.

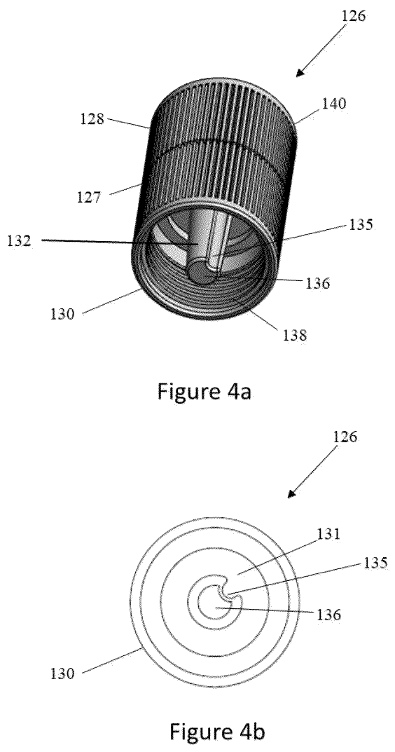

The biological sample collection system 100 also comprises a cap 126 having one or more walls 127 defining a top end 128 and an open end 130. FIG. 4a illustrates a perspective view of the cap 126, and FIG. 4b illustrates a bottom view of cap 126. The cap 126 can be cylindrical and made of a durable material, such as polyethylene, polypropylene or related plastic. The wall 127 of the cap 126 between the open end 130 and top end 128 can be any desired thickness, but should be firm to ensure proper grip by the user for attaching the cap 126 to the tube 102. In another embodiment, the top end 128 of the cap can have features to facilitate removal of the cap 126 from the tube 102 by an automated decapper or liquid-handling robot. In another embodiment, the first end 104 of the tube 102 or the top end 128 of the cap 126 can have features such as tabs and grooves to facilitate the automated assembly of the cap 126 to the tube 102, e.g. when preparing the system for shipment to a user.

The cap 126 has a pusher 132 comprising a first end 134 extending from an inner portion 131 of the cap 126 (see, in particular, FIGS. 2b and 3b) and an opposing second end 136, the second end 136 of the pusher extending a distance from the inner portion 131 of the cap 126 towards the open end 130 of the cap and being configured to engage with the valve 120 of the sample collection reservoir 112. In one embodiment, the distance by which the second end 136 of the pusher 132 extends from the inner portion 131 of the cap 126 is such that the second end 136 extends beyond the utmost end 133 of the open end 130 of the cap 126 (see FIG. 2b), although other arrangements are possible as will be appreciated by the skilled worker and as will become apparent from the description of alternate embodiments below. The cap 126 further has a connection member 138 complementary to the connection member 110 of the tube 102. In the illustrated embodiment, the connection member 138 is internal helical threads on an inner surface of a portion of wall 127 defining the open end 130 of cap 126.

As shown in FIG. 3c, the cap can further have a shoulder 139 configured to engage with a complementary leading surface 141 of the lip 124 of the sample collection reservoir 112, which assists in providing a fluid-tight seal when the connection member 110 of the tube 102 engages with the connection member 138 of the cap 126, as described further below.

The valve 120 has a closed configuration, as best illustrated by FIG. 2c. When the valve 120 is in the closed configuration, the biological sample (e.g. bodily fluid) can be deposited into the sample collection reservoir 112, such as by expectoration from the mouth of a user in embodiments where the biological sample is sputum or saliva. In the closed configuration, the biological sample is retained in the sample collection reservoir 112.

The valve 120 further has an open configuration, as best illustrated by FIG. 3b. When the valve 120 is in the open configuration, the biological sample that has been collected in the sample collection reservoir 112 is released from the sample collection reservoir 112 into the sample storage chamber 108 of the tube 102. This is achieved by closure of the biological sample collection system 100 by engagement of the connection member 110 of the tube 102 with the connection member 138 of the cap 126. Such closure of the biological sample collection system 100 causes the second end 136 of the pusher 132 to engage with the valve 120 of the sample collection reservoir 112, thereby moving the valve 120 from the closed configuration to the open configuration to permit the biological sample to be released from the sample collection reservoir 112 into the sample storage chamber 108 of the tube 102. Specifically, in the embodiment shown, the second end 136 of the pusher 132 engages with and extends through the duck-bill valve 120 to move the valve 120 from the closed configuration to the open configuration.

As best seen in FIGS. 4a and 4b, in one embodiment the pusher 132 comprises a channel or groove 135 in an outer surface thereof, wherein the groove 135 extends along at least a portion of the second end 136 of the pusher along a longitudinal axis of the pusher 132. In another embodiment, the groove 135 extends from the first end 134 of the pusher 132 to the second end 136 of the pusher 132. The groove 135 allows for smooth flow of the collected biological sample from the sample collection reservoir 112 into the sample storage chamber 108 of the tube 102, as it prevents the valve 120 from self-sealing around the pusher 132 which could cause blockage of the flow of sample from the sample collection reservoir 112 into the tube 102.

The sample storage chamber 108 of the tube 102 can contain a stabilizing composition for stabilizing, preserving, and/or facilitating the recovery of biomolecules, such as nucleic acid, from the biological sample. Suitable compositions include those described in, for example, U.S. Pat. No. 7,482,116. Other suitable compositions would be well known to the skilled worker. The stabilizing composition is present in the sample storage chamber 108 of the tube 102 prior to collection of the sample.

As best seen in FIGS. 1, 2a, 2b, 3a, 3b, and 4a, in one embodiment the wall 127 of the cap 126 can define a second open end 140 of the cap 126 which is separated from the open end 130 of the cap 126 by the inner portion 131 of the cap 126, which can form an inner cap wall separating the second open end from the open end of the cap. The cap 126 further has a second connection member 142 disposed at the second open end 140 that is complementary to the connection member 110 of the tube 102. In the illustrated embodiment, the second connection member 142 is internal helical threads on an inner surface of a portion of wall 127 defining the second open end 140 of the cap 126. Thus, the open end 130 and the second open end 140 of the cap 126 can be used interchangeably to engage with and seal the tube 102.

For instance, the biological sample collection system 100 can be shipped in a configuration where the second open end 140 of the cap 126 is connected to the tube 102 by engagement of the connection member 110 of the tube 102 with the connection member 142 disposed at the second open end 140 of the cap 126 (the tube 102 having the sample collection reservoir 112 disposed therein as described above and as shown in FIG. 2c). This configuration closes the biological sample collection system 100 and keeps any contaminants from reaching the interior thereof, while maintaining the valve 120 in a closed configuration such that any sample storage compositions contained within sample storage chamber 108 of the tube 102 are maintained therein. When the biological sample collection system 100 is ready for use, the cap 126 can then be removed from the tube 102 by disengagement of connection members 110 and 142, the biological sample can then be deposited into the sample collection reservoir 112, and the open end 130 of the cap 126 is then connected to the tube 102 by engagement of the connection member 110 of the tube 102 with the connection member 138 disposed at the open end 130 of the cap 126. As described above, this causes the second end 136 of the pusher 132 to engage with the valve 120 of the sample collection reservoir 112, thereby moving the valve 120 from the closed configuration to the open configuration to permit the biological sample to be released from the sample collection reservoir 112 into the sample storage chamber 108 of the tube 102. The biological sample collection system 100 can be maintained in this closed configuration for storage and transport of the biological sample, e.g. to a laboratory environment. As the skilled worker will appreciate, having the sample collection reservoir 112 positioned toward the first end 104 of the tube 102 can minimize or prevent spilling of the stabilization composition and/or sample mixed with the stabilization composition should the tube 102 be accidently knocked over.

The biological sample can then be recovered from the biological sample collection system 100 by disengaging the connection member 110 of the tube 102 from the connection member 138 of the cap 126 to remove the cap 126 from the tube 102. In one embodiment, a manual or automated pipetting device or other implement can then be inserted through the duck-bill valve 120, in order to access the biological sample within the sample storage chamber 108 of the tube 102. In another embodiment, the second end 136 of the pusher 132 can include a flange (not shown) configured to engage with the end 122 of the sample collection reservoir 112 to pull the sample collection reservoir 112 away from the tube 102, thus disengaging the sample collection reservoir 112 from the tube 102 when the cap 126 is removed from the tube 102. In another embodiment, laboratory tweezers or forceps can be used to remove the sample collection reservoir 112 from the tube 102 following removal of the cap 126. In embodiments where a threaded engagement between the sample collection reservoir 112 and the tube 102 is used, the sample collection reservoir 112 can be removed from the tube 102 by dis-engaging the threads (i.e. un-threading).

As the skilled worker will appreciate, and as will become apparent from the description of alternate embodiments below, it is not necessary for the cap 126 of the biological sample collection system 100 to have opposing ends that can be used interchangeably to engage with and seal the tube 102 as outlined above. In another embodiment, the biological sample collection system 100 can have a cap having a single open end with a pusher, and a separate conventional cap can be used to close the biological sample collection system 100 prior to sample collection.

Thus, in one embodiment, there is provided a method of preserving a biomolecule in a biological sample, the method comprising: a) obtaining a biological sample, such as sputum or saliva; b) obtaining the biological sample collection system 100 as described herein; c) placing the biological sample in the sample collection reservoir 112; d) placing the open end 130 of the cap 126 over the first end 104 of the tube 102; e) engaging the connection member 110 of the tube 102 with the connection member 138 of the cap 126, which engages the second end 136 of the pusher 132 with the valve 120 of the sample collection reservoir 112, thereby moving the valve 120 from the closed configuration to the open configuration to permit the biological sample to be released from the sample collection reservoir 112 into the sample storage chamber 108 of the tube 102; and f) mixing the released biological sample with a stabilizing composition in the tube 102 for preserving the biomolecule within the biological sample.

In another embodiment, there is provided a method of preserving a biomolecule in a biological sample, the method comprising: a) obtaining a biological sample; b) obtaining the biological sample collection system 100, wherein the second open end 140 of the cap 126 is connected to the tube 102 via engagement of the second connection member 142 of the cap 126 with the connection member 110 of the tube 102; c) disengaging the second connection member 142 of the cap 126 from the connection member 110 of the tube 102, and removing the cap 126 from the tube 102; d) placing the biological sample in the sample collection reservoir 112; e) placing the open end 130 of the cap 126 over the first end 104 of the tube 102, such that the cap 126 is inverted relative to placement of the cap 126 in step (b); f) engaging the connection member 110 of the tube 102 with the connection member 138 of the cap 126, which engages the second end 136 of the pusher 132 with the valve 120 of the sample collection reservoir 112, thereby moving the valve 120 from the closed configuration to the open configuration to permit the biological sample to be released from the sample collection reservoir 112 into the sample storage chamber 108 of the tube 102; and g) mixing the released biological sample with a stabilizing composition in the tube 102 for preserving the biomolecule within the biological sample.

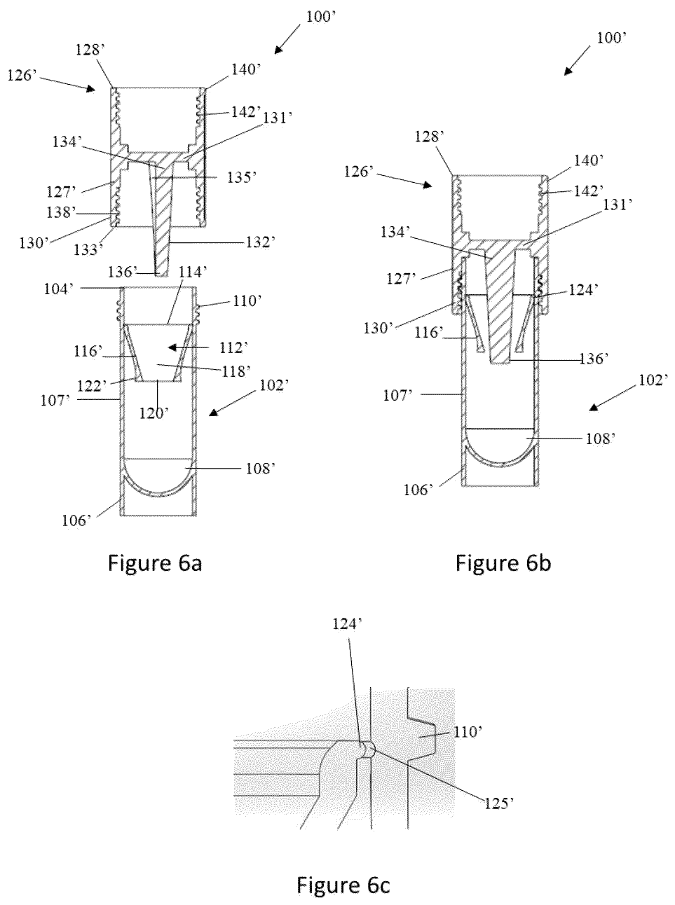

FIGS. 6a-c illustrate a simplified cross-sectional view of an alternate biological sample collection system 100′. In FIGS. 6a-c, elements having reference numbers in common with those of the biological sample collection system 100 operate in a similar manner, and their function will not be repeated here for conciseness. FIG. 6a illustrates the biological sample collection system 100′ with the cap 126′ separated from the containment vessel shown as tube 102′. The sample collection reservoir 112′ is disposed toward the first end 104′ of the tube 102′ and the valve 120′ is in the closed configuration. The sample collection reservoir 112′ is held in place using a ring/snap feature where a lip 124′ of the sample collection reservoir 112′ engages with a groove 125′ on an inner surface of the tube 102′ via a snap-fit connection to hold the sample collection reservoir 112′ in place. FIG. 6b illustrates a simplified cross-sectional view of the biological sample collection system 100′ wherein the biological sample collection system 100′ is closed and the valve 120′ is in the open configuration. FIG. 6c is an enlarged view of the ring/snap feature noted above.

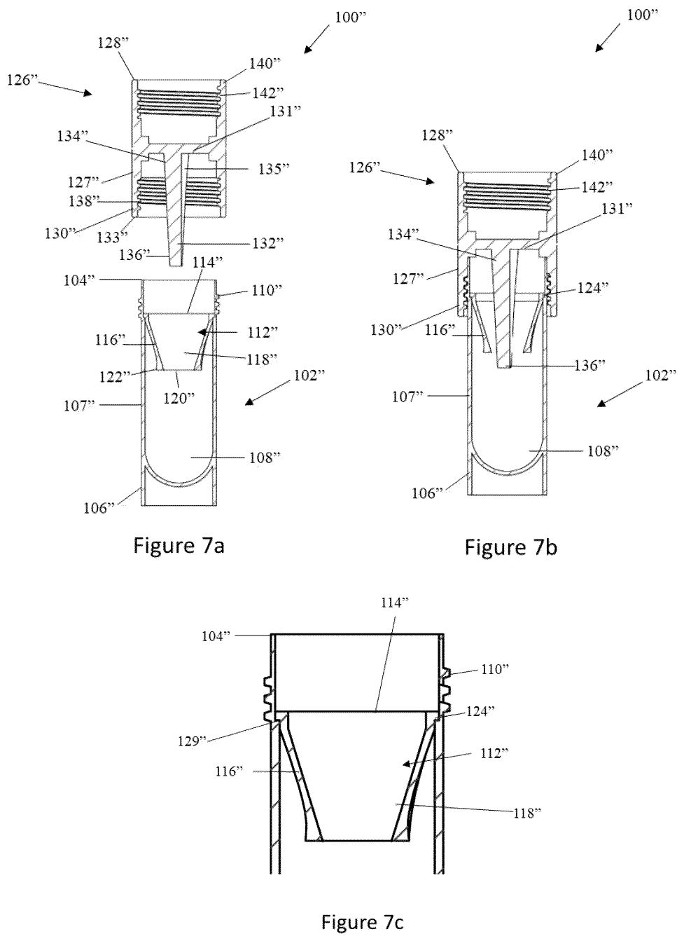

FIGS. 7a-c illustrate a simplified cross-sectional view of an alternate biological sample collection system 100″. In FIGS. 7a-c, elements having reference numbers in common with those of the biological sample collection system 100 operate in a similar manner, and their function will not be repeated here for conciseness. FIG. 7a illustrates a simplified cross-sectional view of the alternate biological sample collection system 100″ with the cap 126″ separated from the containment vessel shown as tube 102″. The sample collection reservoir 112″ is disposed toward the first end 104″ of the tube 102″ and the valve 120″ is in the closed configuration. The sample collection reservoir 112″ is held in place via engagement of a lip 124″ of the sample collection reservoir 112″ with a shoulder 129″ on an inner surface of the tube 102″ via a press-fit connection to hold the sample collection reservoir 112″ in place. FIG. 7b illustrates a simplified cross-sectional view of the biological sample collection system 100″ wherein the biological sample collection system 100″ is closed and the valve 120″ is in the open configuration. FIG. 7c is an enlarged view of the lip/shoulder engagement noted above.

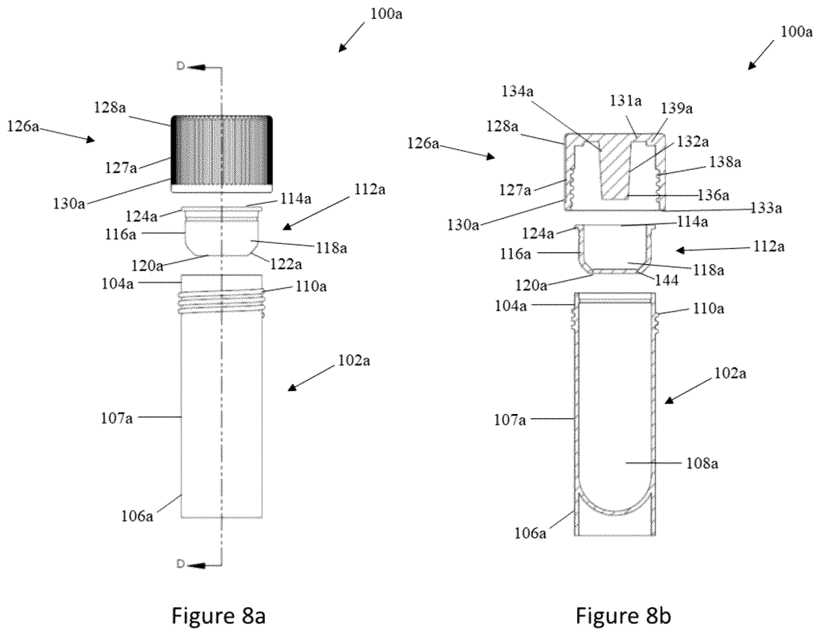

FIGS. 8a-b, 9a-b, 10a-b, 11a-b, 12a-d, and 13a-f illustrate a biological sample collection system 100a and components thereof in accordance with an alternate embodiment of the present application. Components of the biological sample collection system 100a that operate in a similar manner as those described in respect of the biological sample collection system 100 above have been given the same reference numbers with an “a” designation and have similar features described in respect of the biological sample collection system 100 unless otherwise indicated.

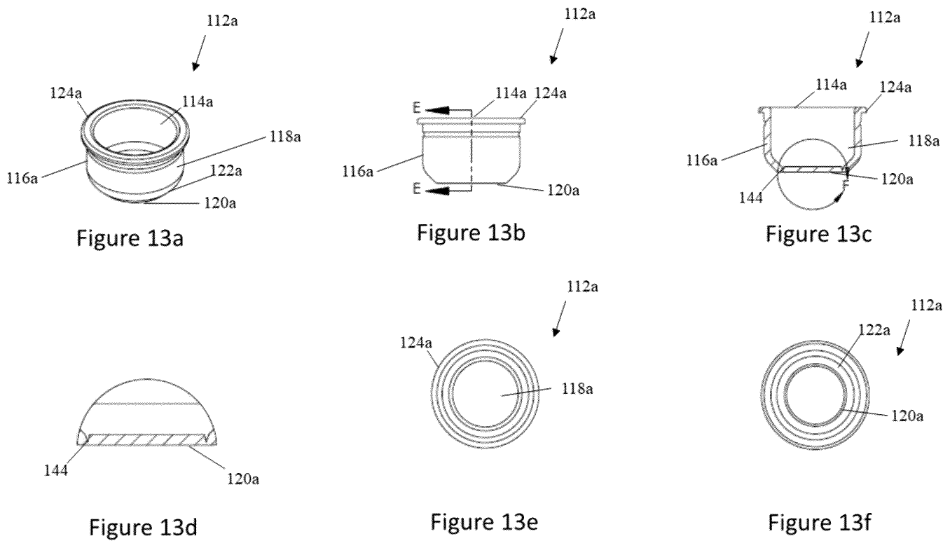

As with the biological sample collection system 100, the biological sample collection system 100a includes a sample collection reservoir 112a that is configured to be disposed toward the first end 104a of the containment vessel shown as tube 102a. However, whereas the valve 120 of the biological sample collection system 100 is a duck-bill valve, the valve 120a of the biological sample collection system 100a can be formed from a flexible material (e.g. rubber, synthetic elastomer) or a rigid plastic (such as low-density polyethylene (LDPE), high-density polyethylene (HDPE), polyethylene terephthalate (PET), etc.) having at least one pre-scored cut 144, wherein the at least one pre-scored cut 144 does not fully penetrate through the flexible material/rigid plastic (see FIGS. 8b, 9b, 10b, 13c and 13d).

Unlike the biological sample collection system 100, the biological sample collection system 100a has a cap 126a with a single open end 130a (see, for example, FIGS. 6b, 9b, 10b, and 11b). Further, the distance by which the second end 136a of the pusher 132a extends from the inner portion 131a of the cap 126a is such that the second end 136a does not extend beyond the utmost end 133a of the open end 130a of the cap 126a (see FIGS. 8b, 9b, 10b, and 11b).

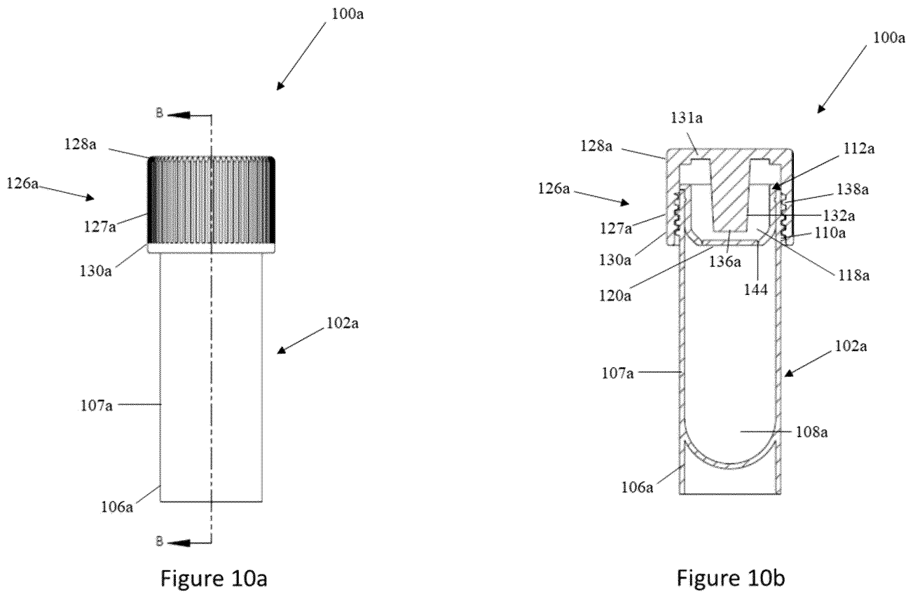

The biological sample collection system 100a has a shipping configuration, as illustrated in FIGS. 10a and 10b, wherein the cap 126a is partly engaged with the tube 102a (and thus the connection member of the tube 110a is partly engaged with the complementary connection member of the cap 138a). This configuration closes the biological sample collection system 100a and keeps any contaminants from reaching the interior thereof, while maintaining the valve 120a in a closed configuration such that any sample storage compositions contained within sample storage chamber 108a of the tube 102a are maintained therein.

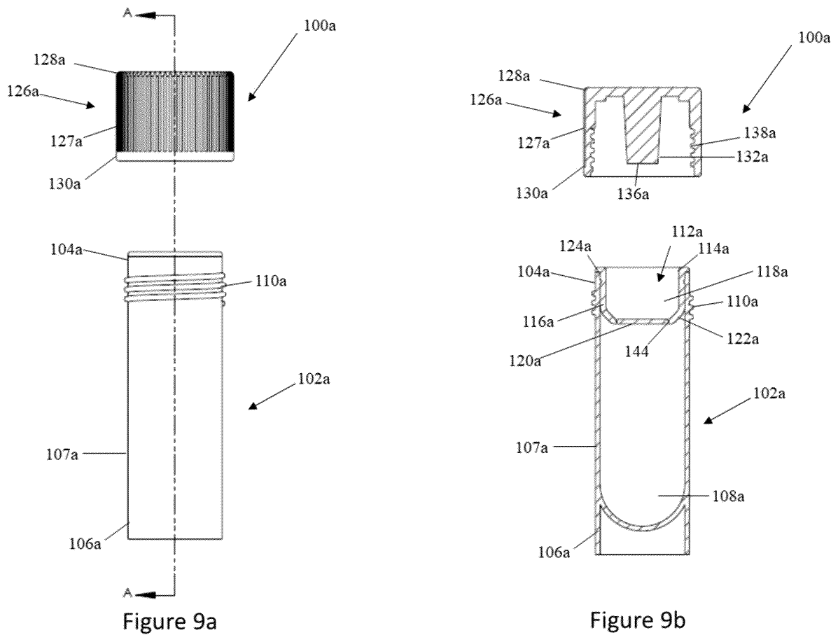

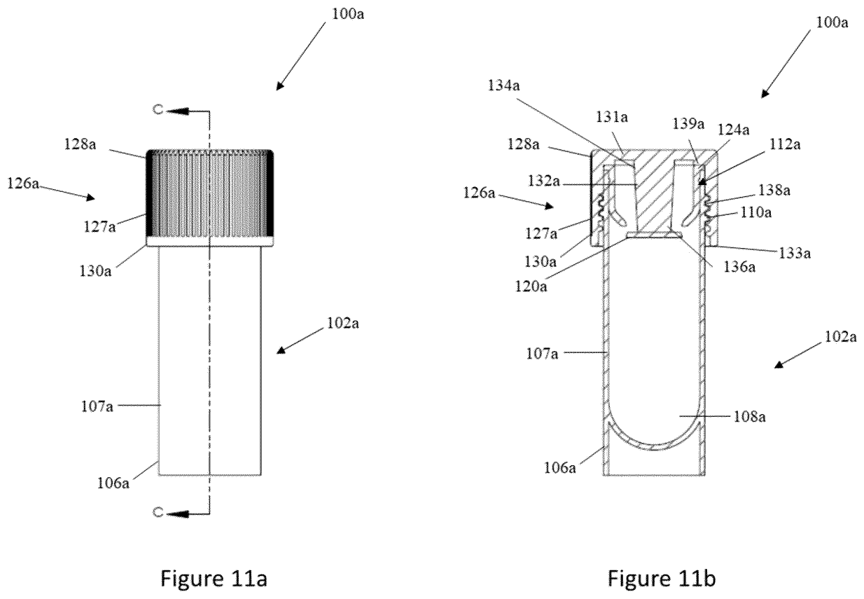

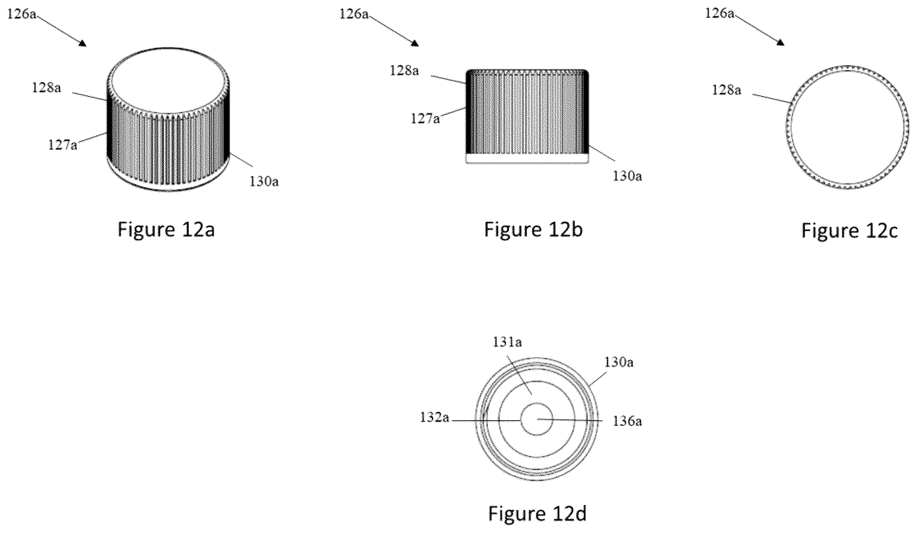

When the biological sample collection system 100a is ready for use, the cap 126a can then be removed from the tube 102a by disengagement of connection members 110a and 138a, the biological sample can then be deposited into the sample collection reservoir 112a. When the valve 120a is in the closed configuration as shown in FIG. 9b, the biological sample (e.g. bodily fluid) can be deposited into the sample collection reservoir 112a, such as by expectoration from the mouth of a user in embodiments where the biological sample is sputum or saliva. In the closed configuration, the biological sample is retained in the sample collection reservoir 112a. Once the biological sample has been collected, the open end 130a of the cap 126a is then fully engaged with the tube 102a by full engagement of the connection member 110a of the tube 102a with the connection member 138a disposed at the open end 130a of the cap 126a. When the biological sample collection system 100a is closed by full engagement of the connection member 110a of the tube 102a with the connection member 138a of the cap 126a, the second end 136a of the pusher 132a engages with the flexible material or rigid plastic in the vicinity of the at least one pre-scored cut 144 to penetrate and extend through the flexible material or rigid plastic to move the valve 120a from the closed configuration (see FIG. 10b) to the open configuration (see FIG. 11b). The valve 120a remains in a permanently open configuration, and the flexible material/rigid plastic can remain partly attached to the remainder of the sample collection reservoir 112a or can be fully detached from same. The biological sample collection system 100a can be maintained in this closed configuration for storage and transport of the biological sample, e.g. to a laboratory environment.

The biological sample can then be recovered from the biological sample collection system 100a by disengaging the connection member 110a of the tube 102a from the connection member 138a of the cap 126a to remove the cap 126a from the tube 102a. In one embodiment, an automated or manual pipetting device or other implement can then be inserted through the permanent opening created by detachment of the flexible material/rigid plastic of valve 120a from the remainder of the sample collection reservoir 112a, in order to access the biological sample within the sample storage chamber 108a of the tube 102a. In another embodiment, the second end 136a of the pusher 132a can include a flange (not shown) configured to engage with the end 122a of the sample collection reservoir 112a to pull the sample collection reservoir 112a away from the tube 102a, thus disengaging the sample collection reservoir 112a from the tube 102a when the cap 126a is removed from the tube 102a. In another embodiment, laboratory tweezers or forceps can be used to remove the sample collection reservoir 112a from the tube 102a following removal of the cap 126a. In embodiments where a threaded engagement between the sample collection reservoir 112a and the tube 102a is used, the sample collection reservoir 112a can be removed from the tube 102a by dis-engaging the threads (i.e. un-threading).

Thus, in one embodiment, there is provided a method of preserving a biomolecule in a biological sample, the method comprising: a) obtaining a biological sample, such as sputum or saliva; b) obtaining the biological sample collection system 100a as described herein; c) placing the biological sample in the sample collection reservoir 112a; d) placing the open end 130a of the cap 126a over the first end 104a of the tube 102a; e) engaging the connection member 110a of the tube 102a with the connection member 138a of the cap 126a, which engages the second end 136a of the pusher 132a with the valve 120a of the sample collection reservoir 112a, thereby moving the valve 120a from the closed configuration to the open configuration to permit the biological sample to be released from the sample collection reservoir 112a into the sample storage chamber 108a of the tube 102a; and f) mixing the released biological sample with a stabilizing composition in the tube 102a for preserving the biomolecule within the biological sample.

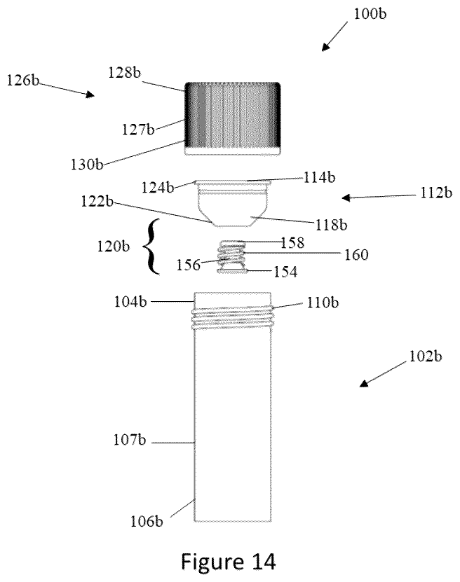

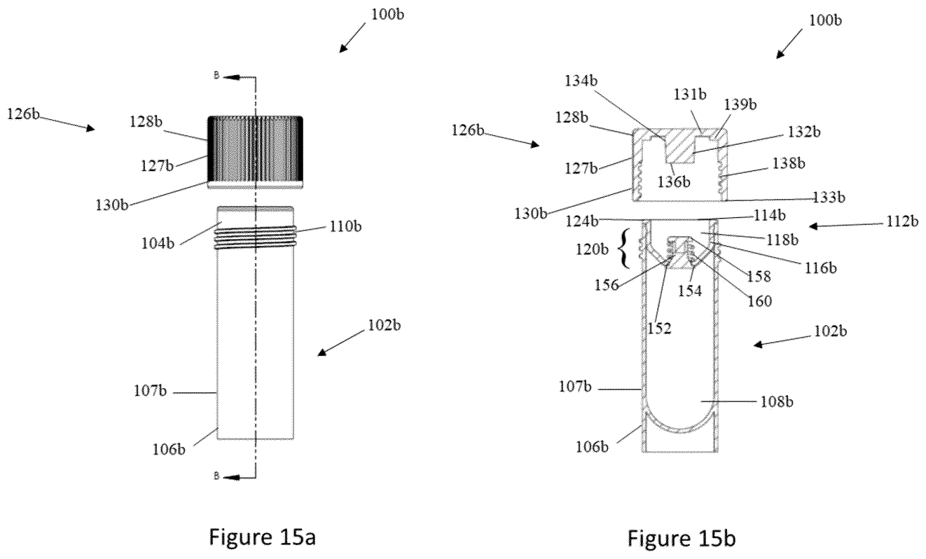

FIGS. 14, 15a-b, 16a-c, 17a-c, 18a-d, and 19a-d illustrate a biological sample collection system 100b and components thereof in accordance with an alternate embodiment of the present application. Components of the biological sample collection system 100b that operate in a similar manner as those described in respect of the biological sample collection system 100 above have been given the same reference numbers with an “b” designation and have similar features described in respect of the biological sample collection system 100 unless otherwise indicated.

As with the biological sample collection system 100, the biological sample collection system 100b includes a sample collection reservoir 112b that is configured to be disposed toward the first end 104b of the containment vessel shown as tube 102b. However, whereas the valve 120 of the biological sample collection system 100 is a duck-bill valve, the valve 120b of the biological sample collection system 100b includes a valve seat 152 and a complementary valve face 154. The valve seat 152 is formed by an outer surface of the sample collection reservoir 112b and defines an opening 153 that is disposed at the end 122b of the sample collection reservoir 112b. The valve face 154 is a leading surface of a flange that engages with the valve seat 152, and the opening 153 is blocked by engagement of the valve seat 152 with the valve face 154. A valve stem 156 extends from the valve face 154 through the opening 153 and is connected to a lock tab 158. The valve 120b further has a spring 160 encircling the valve stem 156 disposed between an inner surface of the sample collection reservoir 112b and the lock tab 158. In the absence of an external force, the spring 160 maintains the engagement between the valve seat 152 and the valve face 154, and the opening 153 is blocked. Those of skill in the art will appreciate that the spring 160 does not need to be a coil spring as shown in the drawings, but rather any spring-like/spring-loaded feature could be used, such as a moulded spring-like feature or even spring-like elements made out of plastic, rubber or other flexible or compressible materials. Valve 120b can be made of any rigid or flexible materials such as rubber, plastics or metals.

Thus, in the biological sample collection system 100b, the valve 120b has a valve seat 152 and a valve face 154, wherein the valve face 154 is movable with respect to the valve seat 152 between the closed configuration, in which the valve seat 152 sealingly engages the valve face 154, and the open configuration, in which the valve face 154 is spaced from the valve seat 152 to allow fluid flow between the valve face 154 and the valve seat 152, and when the biological sample collection system 100b is closed by engagement of the connection member 110b of the tube 102b with the connection member 138b of the cap 126b, the second end 136b of the pusher 132b engages with the valve 120b to move the valve face 154 with respect to the valve seat 152 from the closed configuration to the open configuration.

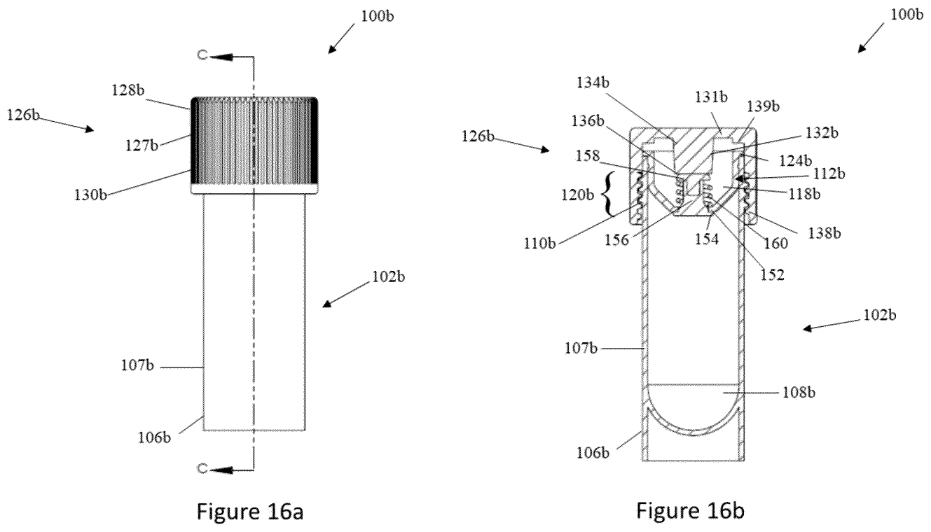

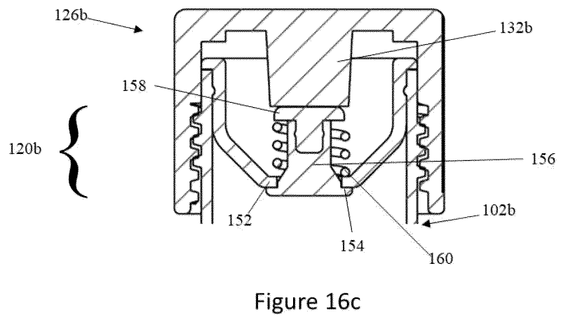

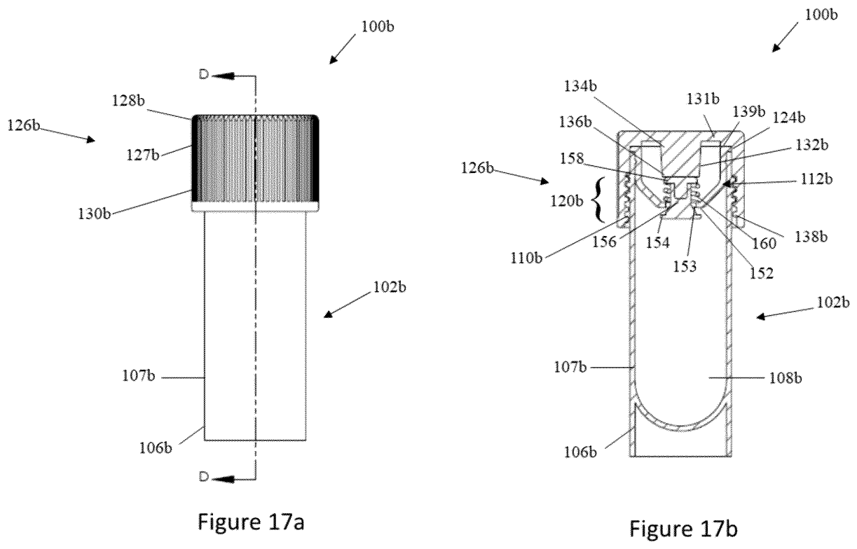

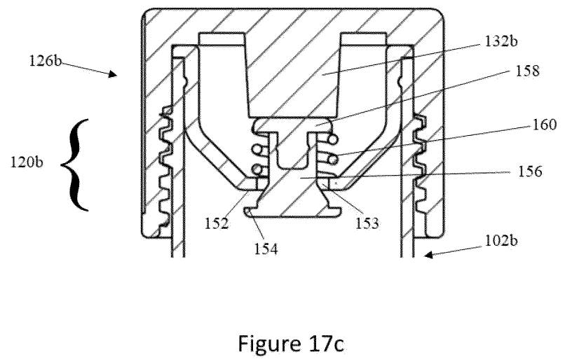

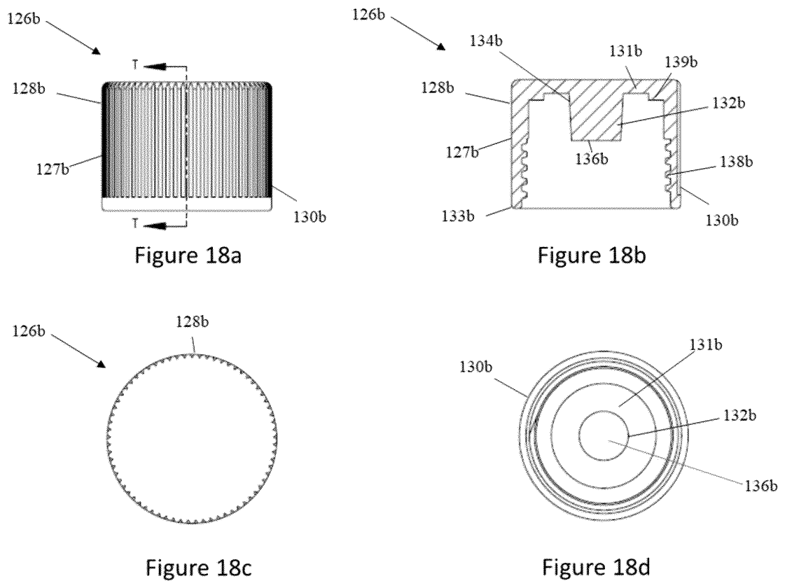

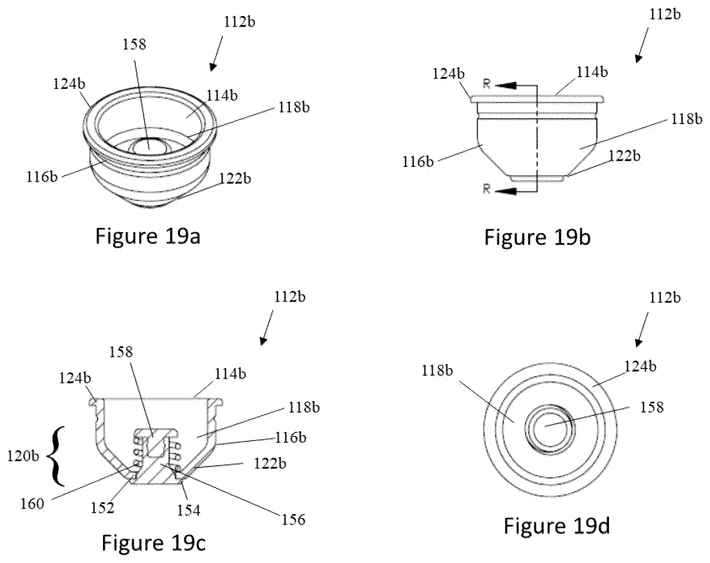

Similar to the biological sample collection system 100a, the biological sample collection system 100b has a cap 126b with a single open end 130b (see, for example, FIGS. 15b, 16b-c, 17b-c, and 18b). The distance by which the second end 136b of the pusher 132b extends from the inner portion 131b of the cap 126b is such that the second end 136b does not extend beyond the utmost end 133b of the open end 130b of the cap 126b.

The biological sample collection system 100b has a shipping configuration, as illustrated in FIG. 16a-c, wherein the cap 126b is partly engaged with the tube 102b (and thus the connection member of the tube 110b is partly engaged with the complementary connection member of the cap 138b). This configuration closes the biological sample collection system 100b and keeps any contaminants from reaching the interior thereof, while maintaining the valve 120b in a closed configuration such that any sample storage compositions contained within sample storage chamber 108b of the tube 102b are maintained therein.

When the biological sample collection system 100b is ready for use, the cap 126b can then be removed from the tube 102b by disengagement of connection members 110b and 138b, and the biological sample can then be deposited into the sample collection reservoir 112b. When the valve 120b is in the closed configuration as shown in FIG. 15b, the biological sample (e.g. bodily fluid) can be deposited into the sample collection reservoir 112b, such as by expectoration from the mouth of a user in embodiments where the biological sample is sputum or saliva. When the valve 120b is in the closed configuration, the biological sample is retained in the sample collection reservoir 112b. Once the biological sample has been collected, the open end 130b of the cap 126b is then fully engaged with the tube 102b by full engagement of the connection member 110b of the tube 102b with the connection member 138b disposed at the open end 130b of the cap 126b. When the biological sample collection system 100b is closed by full engagement of the connection member 110b of the tube 102b with the connection member 138b of the cap 126b, the second end 136b of the pusher 132b engages with and applies force to the lock tab 158 and valve stem 156. The applied force results in axial movement of the valve stem 156 in the direction of the tube 102b and compression of the spring 160, which causes the valve seat 152 to disengage from the valve face 154, thus exposing the opening 153 and allowing flow of the biological sample from the sample collection reservoir 112b into the sample storage chamber 108b of the tube 102b. The biological sample collection system 100b can be maintained in this closed configuration for storage and transport of the biological sample, e.g. to a laboratory environment.

The biological sample can then be recovered from the biological sample collection system 100b by disengaging the connection member 110b of the tube 102b from the connection member 138b of the cap 126b to remove the cap 126b from the tube 102b, at which point laboratory tweezers or forceps can be used to remove the sample collection reservoir 112b from the tube 102b following removal of the cap 126b. In embodiments where a threaded engagement between the sample collection reservoir 112b and the tube 102b is used, the sample collection reservoir 112b can be removed from the tube 102b by dis-engaging the threads (i.e. un-threading).

Thus, in one embodiment, there is provided a method of preserving a biomolecule in a biological sample, the method comprising: a) obtaining a biological sample, such as sputum or saliva; b) obtaining the biological sample collection system 100b as described herein; c) placing the biological sample in the sample collection reservoir 112b; d) placing the open end 130b of the cap 126b over the first end 104b of the tube 102b; e) engaging the connection member 110b of the tube 102b with the connection member 138b of the cap 126b, which engages the second end 136b of the pusher 132b with the valve 120b of the sample collection reservoir 112b, thereby moving the valve 120b from the closed configuration to the open configuration to permit the biological sample to be released from the sample collection reservoir 112b into the sample storage chamber 108b of the tube 102b; and f) mixing the released biological sample with a stabilizing composition in the tube 102b for preserving the biomolecule within the biological sample.

List of elements and reference numbers

| Ref. No. | Element |
|---|---|
| 100/100′/100″/100a/100b | biological sample collection system |
| 102/102′/102″/102a/102b | tube (containment vessel) |
| 104/104′/104″/104a/104b | first end of tube |
| 105 | groove at first end of tube |
| 106/106′/106″/106a/106b | second end of tube |
| 107/107′/107″/107a/107b | wall of tube |
| 108/108′/108″/108a/108b | sample storage chamber of tube |
| 110/110′/110″/110a/110b | connection member of tube |
| 112/112′/112″/112a/112b | sample collection reservoir |
| 114/114′/114″/114a/114b | opening of sample collection reservoir |
| | for receiving a biological sample |
| 116/116′/116″/116a/116b | wall(s) of sample collection reservoir |
| 118/118′/118″/118a/118b | sample receiving area |
| 120/120′/120″/120a/120b | valve |
| 122/122′/122″/122a/122b | end of sample collection reservoir |
| | opposite from opening |
| 124/124′/124″/124a/124b | lip of sample collection reservoir |
| 125′ | groove on inner surface of tube |
| 126/126′/126″/126a/126b | cap |
| 127/127′/127″/127a/127b | wall(s) of cap |
| 128/128′/128″/128a/128b | top end of cap |
| 129″ | shoulder on inner surface of tube |
| 130/130′/130″/130a/130b | open end of cap |
| 131/131′/131″/131a/131b | inner portion of cap |
| 132/132′/132″/132a/132b | pusher |
| 133/133′/133″/133a/133b | utmost end of open end of cap |
| 134/134′/134″/134a/134b | first end of pusher |
| 135/135′/135″ | groove of pusher |
| 136/136′/136″/136a/136b | second end of pusher |
| 138/138′/138″/138a/138b | connection member of cap |
| 139/139a/139b | shoulder of cap |
| 140/140′/140″ | second open end of cap |
| 141 | complementary leading surface of the lip |
| 142/142′/142″ | second connection member of cap |
| 144 | pre-scored cut |
| 152 | valve seat |
| 153 | opening formed by valve seat |
| 154 | valve face |
| 156 | valve stem |
| 158 | lock tab |
| 160 | spring |

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims. The scope of the claims should not be limited to the preferred embodiments set for the description, but should be given the broadest interpretation consistent with the description as a whole.

1-17. (canceled)

18. A biological sample collection system, the system comprising:
a containment vessel comprising:
a first end for receiving a biological sample,
a second end comprising a sample storage chamber, and
a connection member disposed at the first end;
a sample collection reservoir disposed toward the first end of the containment vessel, wherein the sample collection reservoir comprises:
an opening for receiving the biological sample,
one or more walls defining a sample receiving area, and
a valve disposed in the sample receiving area, the valve comprising:
a closed configuration, wherein when the valve is in the closed configuration the biological sample is retained in the sample collection reservoir, and
an open configuration, wherein when the valve is in the open configuration the biological sample is released from the sample collection reservoir into the sample storage chamber of the containment vessel; and
a cap comprising:
a top end,
an open end,
a pusher comprising a first end extending from an inner portion of the cap and an opposing second end, the second end of the pusher extending a distance from the inner portion of the cap towards the open end of the cap and being configured to engage with the valve of the sample collection reservoir, and
a connection member complementary to the connection member of the containment vessel and configured to engage the open end of the cap with the first end of the containment vessel;
wherein,
when the biological sample collection system is closed by engagement of the connection member of the containment vessel with the connection member of the cap, the second end of the pusher engages with the valve of the sample collection reservoir, thereby moving the valve from the closed configuration to the open configuration to permit the biological sample to be released from the sample collection reservoir into the sample storage chamber of the containment vessel.

19. The sample collection system of claim 18, wherein the connection members comprise a threaded connection.

20. The sample collection system of claim 18, wherein engagement of the connection member of the containment vessel with the connection member of the cap creates a fluid-tight seal.

21. The sample collection system of claim 18, wherein the sample collection reservoir comprises at least one marking on said at least one or more walls which corresponds to a fluid volume in the sample receiving area.

22. The sample collection system of claim 18, wherein the sample collection reservoir is funnel-shaped.

23. The sample collection system of claim 18, wherein the valve is a duck-bill valve, and when the biological sample collection system is closed by engagement of the connection member of the containment vessel with the connection member of the cap, the second end of the pusher engages with and extends through the duck-bill valve to move the valve from the closed configuration to the open configuration.

24. The sample collection system of claim 18, wherein the valve is formed from a flexible material or rigid plastic having at least one pre-scored cut, wherein the at least one pre-scored cut does not fully penetrate through the flexible material or rigid plastic, and when the biological sample collection system is closed by engagement of the connection member of the containment vessel with the connection member of the cap, the second end of the pusher engages with the at least one pre-scored cut to penetrate and extend through the flexible material or rigid plastic to move the valve from the closed configuration to the open configuration.

25. The sample collection system of claim 23, wherein the pusher comprises a groove in an outer surface thereof, wherein the groove extends along at least a portion of the second end of the pusher along a longitudinal axis of the pusher.

26. The sample collection system of claim 25, wherein the groove extends from the first end of the pusher to the second end of the pusher.

27. The sample collection system of claim 18, wherein the valve has a valve seat and a valve face, wherein the valve face is movable with respect to the valve seat between the closed configuration, in which the valve seat sealingly engages the valve face, and the open configuration, in which the valve face is spaced from the valve seat to allow fluid flow between the valve face and the valve seat,
and when the biological sample collection system is closed by engagement of the connection member of the containment vessel with the connection member of the cap, the second end of the pusher engages with the valve to move the valve face with respect to the valve seat from the closed configuration to the open configuration.

28. The sample collection system of claim 18, wherein the sample collection reservoir is connected toward the first end of the containment vessel via a press-fit connection between a lip extending from the one or more walls of the sample collection reservoir and a complementary groove disposed at the first end of the containment vessel.

29. The sample collection system of claim 18, wherein the sample collection reservoir is connected toward the first end of the containment vessel via a snap-fit connection between a lip extending from the one or more walls of the sample collection reservoir into a complementary groove disposed on an inner surface of the containment vessel.

30. The sample collection system of claim 18, wherein the sample collection reservoir is connected toward the first end of the containment vessel via a press-fit connection between a lip extending from the one or more walls of the sample collection reservoir and a shoulder on an inner surface of the containment vessel.

31. The sample collection system of claim 18, wherein the sample storage chamber comprises a stabilization composition.

32. The sample collection system of claim 18, wherein the top end of the cap comprises a second open end, the second open end being separated from the open end of the cap by the inner portion of the cap,
the inner portion of the cap comprising an inner cap wall separating the second open end of the cap from the open end of the cap, the first end of the pusher extending from the inner cap wall,
wherein the second open end of the cap comprises a second connection member complementary to the connection member of the containment vessel.

33. A method of preserving a biomolecule in a biological sample, the method comprising:
a) obtaining a biological sample;
b) obtaining the biological sample collection system of claim 18;
c) placing the biological sample in the sample collection reservoir;
d) placing the cap over the first end of the containment vessel;
e) engaging the connection member of the containment vessel with the connection member of the cap, which engages the second end of the pusher with the valve of the sample collection reservoir, thereby moving the valve from the closed configuration to the open configuration to permit the biological sample to be released from the sample collection reservoir into the sample storage chamber of the containment vessel; and
f) mixing the released biological sample with a stabilizing composition in the containment vessel for preserving the biomolecule within the biological sample.

34. A method of preserving a biomolecule in a biological sample, the method comprising:
a) obtaining a biological sample;
b) obtaining the biological sample collection system of claim 32, wherein the second open end of the cap is connected to the containment vessel via engagement of the second connection member of the cap with the connection member of the containment vessel;
c) disengaging the second connection member of the cap from the connection member of the containment vessel, and removing the cap from the containment vessel;
d) placing the biological sample in the sample collection reservoir;
e) placing the open end of the cap over the first end of the containment vessel, such that the cap is inverted relative to placement of the cap in step (b);
f) engaging the connection member of the containment vessel with the connection member of the cap, which engages the second end of the pusher with the valve of the sample collection reservoir, thereby moving the valve from the closed configuration to the open configuration to permit the biological sample to be released from the sample collection reservoir into the sample storage chamber of the containment vessel; and
g) mixing the released biological sample with a stabilizing composition in the containment vessel for preserving the biomolecule within the biological sample.
